(19) United States
(12) Patent Application Publication
(10) Pub. No.: US 2003/0083531 A1
(43) Pub. Date: May 1, 2003

(54) Methods for preparing primary alkyl bromides

(76) Inventors: David T. Erdman, Liberty, MO (US); Jonathan D. Spicher, Parkville, MO (US); Mary F. Lamar, Overland Park, KS (US); David E. Cockrill, Kansas City, MO (US)

Correspondence Address: BAYER CROPSCIENCE LP, 100 BAYER ROAD, PITTSBURGH, PA, 15205, US

(21) Appl. No.: 10/033,330

(22) Filed: Oct. 25, 2001

(51) Int. Cl. C07C 17/20

(52) U.S. Cl. 570/260

(57) ABSTRACT

Methods of converting methyl chloride to methyl bromide include the steps of providing a composition comprising a bromide salt and a liquid; contacting the composition with gaseous methyl chloride and/or methyl chloride dissolved in a water-immiscible solvent; and recovering methyl bromide.

### FIELD OF THE INVENTION

This invention relates to methods of preparing primary alkyl bromides, particularly methyl bromide. The invention also relates to methods of converting methyl chloride to methyl bromide and to methods of reducing the level of bromide salt in aqueous compositions.

### BACKGROUND OF THE INVENTION

Primary alkyl bromides, such as methyl bromide, may be used as fumigants, alkylating agents, or intermediates in the synthesis of other chemical compounds, in particular pharmaceutical or agricultural chemicals.

Methyl bromide may be prepared by treating methanol with hydrobromic acid and sulfuric acid. Unfortunately, such processes required the handling and/or disposing noxious and/or dangerous compounds.

Salt exchange reactions include reactions wherein an alkyl halide is converted to another alkyl halide, such as, for example, the Finkelstein reaction wherein alkyl chlorides or bromides are converted to alkyl iodides using solid potassium or sodium iodide in acetone. Unfortunately, such reactions require the handling or large volumes of acetone, and the equilibrium constants for alkyl chloride/alkyl bromide exchanges in acetone often favor the alkyl chloride.

Hughes et al., J. Chem. Soc., 3173-3175 (1955), teach that the displacement of bromine from alkyl bromides using lithium chloride in dry acetone is only mildly reversible. Starks and Liotta, Phase Transfer Catalysis”, Academic Press, New York, N.Y. (1978), teach the use of phase transfer catalysts in halide exchange reactions in two-phase systems and in homogenous organic reactions, and disclose that the displacement of chloride ion on octyl bromide is faster than bromide ion displacement on octyl chloride.

Handling large volumes of acids may be difficult and/or costly. Additionally, processes using large amounts of sulfuric acid may generate an aqueous waste stream comprising NaSO₄ as well as residual sulfuric acid, and NaSO₄ generally causes more environmental problems than sodium salts such as NaCl or NaBr. Thus, there is a need for facile methods of producing primary alkyl bromides which do not require the use of large volumes of acids.

As handling large volumes of some organic solvents may be difficult and/or costly, there is a need for methods of producing primary alkyl bromides which do not require the use of large volumes of solvents such as acetone.

There is also a need for methods of reducing the level of bromide salts in aqueous compositions.

There is a need for methods of regenerating alkyl bromides from bromide salts which are formed when alkyl bromides, such as methyl bromide, are used as reactants.

### SUMMARY OF THE INVENTION

Accordingly, it is an object of the present invention to obviate problems of the prior art.

It is a further object of the present invention to provide methods of preparing alkyl bromides.

It is also an object of the present invention to provide methods of preparing alkyl bromides which do not require the handling, storage or disposal of large volumes of acetone, hydrobromic acid or sulfuric acid.

It is another object of the present invention to provide methods of removing bromide salts from liquid compositions.

It is a further object of the present invention to provide methods of regenerating alkyl bromides from bromide salts.

These and additional objects are provided by the methods of the invention.

In one embodiment the invention is directed to methods of converting methyl chloride to methyl bromide comprising:

(a) providing an aqueous composition comprising a bromide salt and water;

(b) contacting the aqueous composition with an organic composition comprising an organic solvent, methyl chloride and phase transfer catalyst; and

(c) recovering a product comprising methyl bromide.

In another embodiment the invention is directed to methods of converting methyl chloride to methyl bromide comprising:

(a) providing a composition comprising a bromide salt, water, a first solvent and phase transfer catalyst;

(b) contacting the composition with gaseous methyl chloride and/or methyl chloride dissolved in a second solvent; and

(c) recovering a gaseous product comprising methyl bromide.

In a further embodiment the invention is directed to methods of preparing a primary alkyl bromide comprising:

(a) providing a composition comprising bromide salt, water, solvent and phase transfer catalyst;

(b) contacting the composition with a primary alkyl chloride thereby forming a primary alkyl bromide; and

(c) recovering the primary alkyl bromide.

In another embodiment the invention is directed to methods of reducing the level of bromide salt in an aqueous composition comprising:

(a) providing an aqueous composition comprising a bromide salt and water; and

(b) extracting the aqueous composition with an organic composition comprising an organic solvent, methyl chloride and phase transfer catalyst.

In a further embodiment the invention is directed to methods of converting methyl chloride to methyl bromide comprising:

(a) providing a reaction column oriented substantially vertically along its axis and having an upper portion and a lower portion, wherein the reaction column contains packing suitable for continuous extractions;

(b) introducing an aqueous feed into the upper portion of the reaction column, wherein the aqeous feed comprises water and a bromide salt selected from the group consisting of the alkali metal bromides, alkaline earth metal bromides and combinations thereof;

(c) introducing an organic feed into the lower portion of the reaction column, wherein the organic feed comprises methyl chloride, a water immiscible organic solvent, and a quaternary ammonium salt, quaternary phosphonium salt, or a coordinating catalyst such as crown ether, cryptane, PEGs, TDA-1 and combinations thereof; and

(d) allowing at least one portion of the aqueous feed and at least a portion of the organic feed to contact each other within the reaction column thereby converting at least a portion of the methyl chloride to methyl bromide.

In an additional embodiment the invention is directed to methods of converting methyl chloride to methyl bromide comprising:

(a) providing a reaction column oriented substantially vertically along its axis and having an upper portion and a lower portion, wherein the reaction column contains a solid supported phase transfer catalyst;

(b) introducing an aqueous feed into the upper portion of the reaction column, wherein the aqueous feed comprises water and a bromide salt selected from the group consisting of alkali metal bromides, alkaline earth metal bromides and combinations thereof;

(c) introducing an organic feed into the lower portion of the reaction column, wherein the organic feed comprises methyl chloride and a water immiscible organic solvent; and

(d) allowing at least a portion of the aqueous feed and at least a portion of the organic feed to contact each other within the reaction column in the presence of the solid supported salt phase transfer catalyst, thereby converting at least a portion of the methyl chloride to methyl bromide.

These and additional aspects, objects and advantages of the invention are more fully described in the following detailed description.

### DETAILED DESCRIPTION

The present invention is directed to the preparation of primary alkyl bromides in the presence of a liquid. The present invention is also directed to methods of reducing the level of bromide salts in an aqueous solution, and methods of regenerating alkyl bromides.

The primary alkyl bromides are prepared by providing a composition comprising bromide salt and a liquid; contacting the composition with a primary alkyl chloride thereby forming a primary alkyl bromide; and recovering the primary alkyl bromide. Methods in accordance with the invention may be used to regenerate alkyl bromides from bromide salts which are formed when alkyl bromides are used as reactants.

Methods in accordance with the invention avoid the use of large volumes of acids and/or organic solvents such as acetone. Thus any resulting aqueous waste stream may be easily handled and/or treated.

While not being bound by theory, it is believed that the reaction proceeds as set forth below:

R—X+ZBr→R—Br+ZX

wherein R is a primary alkyl, preferably a C₁-C₂₀ primary alkyl, more preferably a C₁-C₅ alkyl; even more preferably a C₁-C₂ alkyl; X is a halogen, preferably a halogen other than Br, more preferably Cl; Z is a cation such hydrogen, sodium, potassium, lithium, magnesium, calcium or copper, preferably hydrogen, sodium, potassium or lithium.

Suitable bromide salts include hydrogen bromide, alkali metal bromides, alkaline earth metal bromides, organic quaternary Group V bromides and combinations thereof. In one embodiment the bromide salt is selected from the group consisting of sodium bromide, potassium bromide, lithium bromide, hydrogen bromide and combinations thereof, while in another embodiment the bromide salt is selected from the group consisting of organic quaternary bromides and mixtures thereof.

Suitable organic quaternary bromides may be selected from the group consisting of organic quaternary nitrogen bromide, organic quaternary phosphorous bromide, organic quaternary arsenic bromide, organic quaternary antimony bromide, organic quaternary bismuth bromide and combinations thereof, preferably the organic quaternary bromide selected from the group consisting of organic quaternary nitrogen bromide, organic quaternary phosphorous bromide, and combinations thereof. The organic groups of the organic quaternary bromide are each independently selected from benzyl or monovalent hydrocarbon radicals having from 1 to 20 carbon atoms. Suitable organic quaternary bromides include tri(octyl)methylammonium bromide, tetra(octyl)ammonium bromide, benzyl tributyl ammonium bromide, tetrabutylammonium bromide, tetrabutyl phosphonium bromide and combinations thereof.

The bromide salt may be present in a mixture with salts other than bromide salts, such as sodium sulfate and sodium chloride. The bromide salt may be present in the liquid at a concentration of from about 1% to about 50%, preferably from about 5% to about 35%, by weight.

The liquid may comprise water and/or organic solvents. Preferred organic solvents include water-immiscible organic solvents. The amount of organic solvent, preferably water-immiscible organic solvent, in the liquid may be a weight of organic solvent of from about 2 times to about 50 times, preferably from about 5 times to about 20 times, the weight of the primary alkyl chloride.

The preparation of the primary alkyl bromide may occur in the substantial absence of water, or in the substantial absence of water-immiscible organic solvent. As used herein “substantial absence of water” is intended to mean to liquid comprises less than 10%, by weight, water, while “substantial absence of water-immiscible organic solvent” is intended to mean to liquid comprises less than 10%, by weight, water-immiscible organic solvent. In one embodiment the preparation of the primary alkyl bromide occurs in the substantial absence, preferably the absence, of acetone.

The water-immiscible organic solvent may be selected from the group consisting of C₅-C₂₀ aliphatic and cycloaliphatic hydrocarbon solvents, mono- and polycyclic aromatic solvents, di(C₁-C₄ alkyl)form-amides, ketones such as C₄-C₈ ketones, and combinations thereof.

As used herein “alkyl” refers to both linear and branched chain alkyls. As used herein “C₅-C₂₀ aliphatic and cycloaliphatic hydrocarbon solvents” refer to hydrocarbons having from 5 to 20 carbons atoms in the hydrocarbon backbone and which may be substituted or unsubstituted. Suitable substituents include halides, ethers, esters, cyano, amides, alkenes, alkynes and combinations thereof.

As used herein “mono- and polycyclic aromatic solvents” refer to solvents containing one or more cyclic moiety. The cyclic moiety may contain a heteroatom in the ring, and may be substituted or unsubstituted. As used herein, “heteroatom” refers to atoms other than carbon, such as nitrogen, oxygen and sulfur. Suitable substituents include C₁-C₉ alkyl groups, nitro, halides, cyano, esters, ethers, amides and combinations thereof. Suitable mono- and polycyclic aromatic solvents include benzene, toluene, pyridine, furan, thiophene, piperidine.

Typical water-immiscible organic solvents include benzene, toluene, xylene, methyl cyclohexane, methyl iso-butylketone, ethyl acetate, methyl-tert-butyl ether, chloroform, dichloromethane, ethyl ether, hexane, heptane, octanol and combinations thereof. Preferred water immiscible organic solvents include toluene and methyl iso-butylketone.

As used here, “primary alkyl chloride” is intended to refer to compounds having a chloride on a terminal alkyl carbon, and is intended to include arylalkyls having a chloride on a terminal alkyl carbon. The primary alkyl chloride molecules generally have from about 1 to about 20, preferably from about 1 to about 5, more preferably from about 1 to about 2, carbon atoms. Suitable primary alkyl chlorides include 1-chlorobutane, 1-chloroethane, methyl chloride, benzyl chloride, or mixtures thereof. Preferably the primary alkyl chloride is methyl chloride.

The bromide salt is present in an amount to provide a molar equivalent ratio of bromide ions to primary alkyl chloride of from about 1:10 to about 10:1. In one preferred embodiment the the primary alkyl chloride is methyl chloride, and the bromide salt is present in an amount to provide a molar equivalent ratio of bromide ions to methyl chloride of from about 1:10 to about 10:1

The preparation of the primary alkyl bromide may occur in the presence or absence of a phase transfer catalyst. In one embodiment of the invention the step of contacting the composition comprising the bromide salt and liquid with the primary alkyl chloride, preferably methyl chloride, occurs in the substantial absence of phase transfer catalyst. As used herein “substantial absence of phase transfer catalyst” is intended to mean that the resulting reaction mixture comprising the bromide salt, liquid and primary alkyl chloride comprises or no more than about 0.5%, preferably less than about 0.5%, by weight phase transfer catalyst.

In another embodiment of the invention the composition comprising the bromide salt and the liquid further comprises a phase transfer catalyst, preferably the composition comprises a bromide salt selected from the group consisting of alkali metal bromides, alkaline earth metal bromides and combinations thereof, a liquid comprising a water-immiscible organic solvent, and a phase transfer catalyst.

Suitable phase transfer catalysts may be selected from the group consisting of organic quaternary Group V salts, crown ethers, cryptanes, polyalkylene glycols such as poly(C₂-C₄ alkylene glycols), tris[2-(2-methoxyethoxy)-ethyl]amine (TDA-1) and combinations thereof. Suitable polyalkylene glycols include polyethylene glycols (PEGs). Amines or phosphines which are quaternized under the reaction conditions may also be used as phase transfer catalysts.

In one embodiment the phase transfer catalyst is an organic quaternary Group V salt selected from the group consisting of organic quaternary nitrogen salts, organic quaternary phosphorous salts, organic quaternary arsenic salts, organic quaternary antimony salts, organic quaternary bismuth salts, and combinations thereof. The organic groups of the organic quaternary Group V salt may selected from benzyl and monovalent hydrocarbon radicals, preferably from benzyl or monovalent hydrocarbon radicals having from 1 to 20 carbon atoms.

In one embodiment of the invention the catalyst is a quaternary ammonium or phosphonium salt of the structure:

R¹R²R³R⁴Q⁺X⁻

wherein R¹, R2, R3 and R⁴ are each independently selected from benzyl and alkyl, preferably from benzyl and monovalent hydrocarbon radicals having from 1 to 20 carbon atoms; Q is nitrogen or phosphorus; and Z is an anion such as bromide, chloride, sulfate or hydroxy. Suitable organic quaternary salts include tri(octyl)methylammonium salts, tetra(octyl)ammonium salts, benzyl tributyl ammonium salts, tetrabutylammonium salts, tetrabutyl phosphonium salts and combinations thereof.

In one embodiment of the invention the phase transfer catalyst, preferably an organic quaternary Group V salt, is attached to a solid support, referred to herein as a “solid supported” catalyst. The solid supported catalyst may be used at packing in a reactor column. Preferably the solid support is an organic polymer. Suitable organic polymers include polyester polymers, polyvinyl polymers, polystyrene polymers, poly(meth)acrylate polymers and combinations thereof

In one embodiment of the invention the catalyst is a quaternary ammonium or phosphonium salt of the structure:

R¹R²R³R⁴Q^{+X}³¹

wherein R¹, R² and R³ are each independently selected from benzyl and alkyl, preferably from benzyl and monovalent hydrocarbon radicals having from 1 to 20 carbon atoms; R⁴ is a polymer resin of the gel or macroporous/macro-reticular type; Q is nitrogen or phosphorus; and Z is an anion such as bromide, chloride, sulfate or hydroxy. Such phase catalysts include quaternary ammonium salt substituted polymer ion-exchange resins, such as macroreticular resins and macroporous highly crosslinked resins. Such resins include Amberjet™ 4200(Cl), Amberlite® IRA-400(Cl), IFA-410, and IRA-743 gel resins and Amberlite® IRA-900 macroreticular resins.

The phase transfer catalyst is present in an amount sufficient to catalyze the conversion of the primary alkyl chloride to the primary alkyl bromide. For example the phase transfer catalyst may be present in an amount of from about 0.5 to about 50, preferably from about 1 to about 25, mole percent based on total moles of added primary alkyl chloride. In one embodiment of the invention the phase transfer catalyst is an organic quaternary Group V salt and the primary alkyl chloride is methyl chloride, and the organic quaternary Group V salt is present in an amount of from about 0.5 to about 50 mole percent based on total moles of methyl chloride added.

The methods in accordance with the present invention may be performed at any pressure and temperature sufficient for the desired reaction to occur. Generally the methods are performed at a temperature of from about 40° C. to about 200° C., preferably from about 60° C. to about 80° C., and at pressure of from about 0 to about 20 atmospheres.

The conversion of the primary alkyl chloride to primary alkyl bromide may be performed batchwise, semi-batchwise or continuously, and gases produced during the conversion may be purified by continuous or batchwise distillation, or by pressure swing absorption methods.

Any suitable reactor may be used, including fixed-bed reactors having an immobilized phase transfer catalyst. For example, one embodiment of the invention comprises:

(a) providing a reaction column oriented substantially vertically along its axis and having an upper portion and a lower portion, wherein the reaction column contains a solid supported phase transfer catalyst;

(b) introducing an aqueous feed into the upper portion of the reaction column, wherein the aqueous feed comprises water and a bromide salt selected from the group consisting of alkali metal bromides, alkaline earth metal bromides and combinations thereof;

(c) introducing an organic feed into the lower portion of the reaction column, wherein the organic feed comprises methyl chloride and a water immiscible organic solvent; and

(d) allowing at least a portion of the aqueous feed and at least a portion of the organic feed to contact each other within the reaction column in the presence of the solid supported salt phase transfer catalyst, thereby converting at least a portion of the methyl chloride to methyl bromide.

A further embodiment of the invention comprises:

(a) providing a reaction column oriented substantially vertically along its axis and having an upper portion and a lower portion, wherein the reaction column contains packing suitable for continuous extractions;

(b) introducing an aqueous feed into the upper portion of the reaction column, wherein the aqueous feed comprises water and a bromide salt selected from the group consisting of the alkali metal bromides, alkaline earth metal bromides and combinations thereof;

(c) introducing methyl chloride gas into the lower portion of the reaction column; and

(d) allowing at least one portion of the aqueous feed and at least a portion of the methyl chloride to contact each other within the reaction column thereby converting at least a portion of the methyl chloride to methyl bromide.

Another embodiment of the invention comprises:

(a) providing a reaction column oriented substantially vertically along its axis and having an upper portion and a lower portion, wherein the reaction column contains packing suitable for continuous extractions;

(b) introducing an organic feed into the upper portion of the reaction column, wherein the organic feed comprises a water immiscible organic solvent and a bromide salt selected from the group consisting of quaternary ammonium salts, quaternary phosphonium salts, alkali metal bromides, alkaline earth metal bromides and combinations thereof, and a catalyst selected from the group consisting of crown ethers, cryptanes, PEGs, TDA-1, and combinations thereof;

(c) introducing methyl chloride gas into the lower portion of the reaction column; and

(d) allowing at least one portion of the organic feed and at least a portion of the methyl chloride feed to contact each other within the reaction column thereby converting at least a portion of the methyl chloride to methyl bromide

One embodiment of the invention comprises:

(a) providing a reaction column oriented substantially vertically along its axis and having an upper portion and a lower portion, wherein the reaction column contains packing suitable for continuous extractions;

(b) introducing an aqueous feed into the upper portion of the reaction column, wherein the aqueous feed comprises water and a bromide salt selected from the group consisting of the alkali metal bromides, alkaline earth metal bromides and combinations thereof;

(c) introducing an organic feed into the lower portion of the reaction column, wherein the organic feed comprises methyl chloride, a water immiscible organic solvent, and a quaternary ammonium salt, quaternary phosphonium salt, or a coordinating catalyst such as crown ether, cryptane, PEGs, TDA-1 and combinations thereof; and

(d) allowing at least one portion of the aqueous feed and at least a portion of the organic feed to contact each other within the reaction column thereby converting at least a portion of the methyl chloride to methyl bromide.

Methods in accordance with the present invention provide good recoveries of the bromide salt. Generally at least about 30%, preferably at least about 50%, more preferably at least about 60%, by weight, of the bromide salt is converted to primary alkyl bromide. In one embodiment, the primary alkyl bromide is methyl bromide, and at least about 50%, preferably at least about 75%, more preferably at least about 80%, even more preferably at least about 90%, of the bromide salt is converted to methyl bromide. In one embodiment of the invention any unreacted primary alkyl chloride is recovered and may be used recycled in a subsequent bromide salt conversion.

Excessive hydrolysis of the primary alkyl bromide product can be avoided using methods of the present invention. Generally no more than about 25%, preferably no more than about 10%, more preferably no more than about 5%, by weight, of the primary alkyl bromide undergoes hydrolysis.

In one embodiment of the invention the step of contacting a composition comprising the bromide salt and the liquid with the primary alkyl chloride, preferably methyl chloride, occurs in the substantial absence of phase transfer catalyst and water-immiscible organic solvent, while in another embodiment the bromide salt is a quaternary bromide salt and the step of contacting a composition comprising the bromide salt and the liquid with the primary alkyl chloride, preferably methyl chloride, occurs in the substantial absence of water, alkali metal bromides and alkaline earth metal bromides. In yet a further embodiment of the invention the composition comprises a water-immiscible organic solvent and a bromide salt selected from the group consisting of organic quaternary bromides and combinations thereof, and the step of contacting the composition with methyl chloride occurs in the substantial absence of water, alkali metal bromides and alkaline earth metal bromides.

In another embodiment of the invention contacting a composition comprising a bromide salt, a phase catalyst and a liquid with methyl chloride results in the formation of a heterogeneous mixture comprising the methyl chloride, the bromide salt, the phase transfer catalyst, and the liquid. In a further embodiment the phase catalyst is an organic quaternary Group V salt, which may optionally be attached to a solid support, while the bromide salt is selected from alkali metal bromides, alkaline earth metal bromides and combinations thereof.

In one embodiment of the invention an aqueous composition comprising bromide salt is extracted with an organic solvent in the presence of a phase transfer catalyst. The resulting organic phase comprising organic solvent, bromide salt and phase transfer catalyst is then contacted with gaseous primary alkyl chloride and/or primary alkyl chloride dissolved in a water-immiscible solvent. Primary alkyl bromides are produced. While not being bound by theory, it is believed that during the extraction of the aqueous composition with the organic solvent the bromide ion exchanges with the anion of the phase transfer catalyst, and during contact with the primary alkyl chloride the bromide of the quaternary compound exchanges with the chloride.

Preferably the primary alkyl chloride is gaseous methyl chloride and/or methyl chloride dissolved in a water-immiscible solvent. The bromide salt is typically selected from the group consisting of alkali metal bromides, alkaline earth metal bromides, organic quaternary bromides and combinations thereof. The phase transfer catalyst is preferably selected from quaternary ammonium compounds, quaternary phosphonium compounds and combinations thereof. While not being bound by theory the reaction is believed to occur as set forth below:

ZBr(aqueous phase)+QuatX(organic phase)→ZX(aqueous phase)+QuatBr(organic phase) R—X+QuatBr→R—Br+QuatX

wherein R is a primary alkyl, preferably a C₁-C₂₀ primary alkyl, more preferably a C₁-C₅ alkyl; even more preferably a C₁-C₂ alkyl; X is a halogen, preferably a halogen other than Br, more preferably Cl; Z is a cation such hydrogen, sodium, potassium, lithium, magnesium, calcium or copper, preferably hydrogen, sodium, potassium or lithium; and Quat is a quaternary ammonium or quaternary phosphonium compound.

Methods in accordance with the present invention may be used to reduce the level of bromide salt in aqueous compositions. Accordingly, the method may comprise converting at least a portion of a bromide salt in an aqueous composition to primary alkyl bromides, and removing the primary alkyl bromides from the aqueous composition. The primary alkyl chloride may be selected from the group consisting of 1-chlorobutane, 1-chloroethane, methyl chloride, benzyl chloride, and combinations thereof, preferably the primary alkyl chloride in methyl chloride. The bromide salt may be selected from the group consisting of alkali metal bromides, alkaline earth metal bromides, organic quaternary bromides and combinations thereof. A phase transfer catalyst, preferably a phase transfer catalyst selected from the group consisting of organic quaternary Group V salts, crown ethers, cryptanes, polyalkylene glycols such as poly(C₂-C₄ alkylene glycols), tris[2-(2-methoxyethoxy)-ethyl]amine and combinations thereof, may be added to the composition at a level sufficient to facilitate phase transfer.

The bromide salt may be present in the aqueous composition at a level of from about 1% to about 50%, by weight of the composition, preferably at a level of from about 5% to 35%, by weight of the composition. The process may be performed at a temperature of at least about 40° C., preferably from about 40° C. to about 200° C., more preferably from about 50° C. to about 90° C., and at a pressure of from about 0 to about 20 atmospheres.

Generally at least about 30%, preferably at least about 50%, more preferably at least about 60%, by weight, of the bromide salt is removed from the aqueous compositions in a single batch reaction. Repetition of the batch reactions, or a continuous extractor setup, would allow the removal of at least about 80%, preferably at least about 90%, more preferably at least about 95%, by weight, of the bromide salts.

In one embodiment of the invention a method of reducing the level of bromide salt in an aqueous composition comprising bromide salt and water, the method comprising the step of extracting the aqueous composition with an organic composition comprising an organic solvent and a phase transfer catalyst to obtain an organic phase comprising the organic solvent, phase transfer catalyst and bromide salt. The primary alkyl chloride may be methyl chloride, and the phase catalyst is selected from the group consisting of organic quaternary Group V salts, crown ethers, polyalkylene glycols such as polyethylene glycols, cryptanes, tris[2-(2-methoxyethoxy)-ethyl]amine and combinations thereof. The method may further comprise the step of heating the organic phase in the presence of an primary alkyl chloride. The organic phase may to a temperature of at least about 40° C., preferably of from about 40° C. to about 200° C.

Throughout the examples and the present specification, parts and percentages are by weight unless otherwise specified. The following example is illustrative only and is not intended to limit the scope of the methods of the invention as defined by the claims.

### EXAMPLES

### EXAMPLE 1

About 100 g of toluene, about 51 g of sodium bromide, about 10 g of trioctylmethylammonium chloride and about 5 g of methyl chloride are agitated together at about 78° C. for about 4 hours. The conversion of methyl chloride to methyl bromide is about 95% complete.

### EXAMPLE 2

About 100 g of methylisobutylketone, about 50 g of water, about 10 g of sodium bromide, and about 5 g of methyl chloride are agitated together at about 78° C. for about 4 hours. The conversion of methyl chloride to methyl bromide is about 75% complete.

### EXAMPLE 3

About 2500 g of toluene, about 500 g of an aqueous solution of 20% sodium bromide in water, 500 g of Amberlite™ 410 gel resin and about 500 g methyl chloride are agitated together at 68° C. and 80 psi for about 60 minutes. About 70% of the sodium bromide is converted to methyl bromide, and/or is removed from the aqueous phase.

### EXAMPLE 4

About 2500 g of toluene, about 500 g of an aqueous solution of 20% sodium bromide in water, 500 g of Amberlite™ 900 macroreticular resin and about 500 g methyl chloride are agitated together at 68° C. and 80 psi for about 60 minutes. About 60% of the sodium bromide is converted to methyl bromide, and/or is removed from the aqueous phase.

Additional embodiments and modifications within the scope of the claimed invention will be apparent to one of ordinary skill in the art. Accordingly, the scope of the present invention shall be considered in terms of the following claims, and is understood not to be limited to the details of the methods described in the specification.

What is claimed is:

1. A method of converting methyl chloride to methyl bromide comprising:
(a) providing an aqueous composition comprising a bromide salt and water;
(b) contacting the aqueous composition with an organic composition comprising an organic solvent, methyl chloride and phase transfer catalyst; and
(c) recovering a product comprising methyl bromide.

2. A method according to claim 1, wherein the phase transfer catalyst is selected from the group consisting of organic quaternary Group V salts, crown ethers, cryptanes, polyalkylene glycols, amines and phosphines which are quaternized under the method conditions, and combinations thereof.

3. A method according to claim 1, wherein the phase transfer catalyst is selected from quaternary ammonium compounds, quaternary phosphonium compounds and combinations thereof.

4. A method according to claim 1, wherein the phase transfer catalyst is attached to a solid support.

5. A method according to claim 1, wherein the organic solvent is a water-immiscible solvent.

6. A method of converting methyl chloride to methyl bromide comprising:
(a) providing a composition comprising a bromide salt, water, a first solvent and phase transfer catalyst;
(b) contacting the composition with gaseous methyl chloride and/or methyl chloride dissolved in a second solvent; and
(c) recovering a gaseous product comprising methyl bromide.

7. A method according to claim 6, wherein the bromide salt is selected from the group consisting of alkali metal bromides, alkaline earth metal bromides, organic quaternary bromides and combinations thereof.

8. A method according to claim 6, wherein the first and second solvents are the same or different water-immiscible solvents.

9. A method according to claim 6, wherein the composition comprise an amount of bromide salt to provide a molar equivalents ratio of bromide ions to methyl chloride is at least about 1:1.

10. A method according to claim 6, wherein the phase transfer catalyst is selected from the group consisting of organic quaternary Group V salts, crown ethers, cryptanes, polyalkylene glycols, amines and phosphines which are quaternized under the method conditions, and combinations thereof.

11. A method according to claim 10, wherein the anion of the organic quaternary Group V salt is selected from chloride, bromide, hydroxy, sulfate and combinations thereof.

12. A method according to claim 6, wherein the phase transfer catalyst is attached to a solid support.

13. A method according to claim 6, wherein the phase transfer catalyst in a solid supported organic quaternary Group V salt.

14. A method of preparing a primary alkyl bromide comprising:
(a) providing a composition comprising bromide salt, water, solvent and phase transfer catalyst;
(b) contacting the composition with a primary alkyl chloride thereby forming a primary alkyl bromide; and
(c) recovering the primary alkyl bromide.

15. A method according to claim 14, wherein the primary alkyl chloride is selected from the group consisting of 1-chlorobutane, 1-chloroethane, methyl chloride, benzyl chloride, and combinations thereof.

16. A method according to claim 14, wherein the bromide salt is selected from the group consisting of alkali metal bromides, alkaline earth metal bromides, organic quaternary bromides and combinations thereof.

17. A method according to claim 14, wherein the phase transfer catalyst is selected from the group consisting of organic quaternary Group V salts, crown ethers, cryptanes, polyalkylene glycols and combinations thereof.

18. A method of reducing the level of bromide salt in an aqueous composition comprising:
(a) providing an aqueous composition comprising a bromide salt and water; and
(b) extracting the aqueous composition with an organic composition comprising an organic solvent, methyl chloride and phase transfer catalyst.

19. A method according to claim 18, further comprising:
(b) recovering a product comprising methyl bromide.

20. A method according to claim 18, wherein the phase transfer catalyst is selected from the group consisting of unsupported phase transfer catalysts, phase transfer catalyst attached to a solid support, and combinations thereof.

21. A method of converting methyl chloride to methyl bromide comprising:
(a) providing a reaction column oriented substantially vertically along its axis and having an upper portion and a lower portion, wherein the reaction column contains packing suitable for continuous extractions;
(b) introducing an aqueous feed into the upper portion of the reaction column, wherein the aqueous feed comprises water and a bromide salt selected from the group consisting of the alkali metal bromides, alkaline earth metal bromides and combinations thereof;
(c) introducing an organic feed into the lower portion of the reaction column, wherein the organic feed comprises methyl chloride, a water immiscible organic solvent, and a quaternary ammonium salt, quaternary phosphonium salt, or a coordinating catalyst such as crown ether, cryptane, PEGs, TDA-1 and combinations thereof; and
(d) allowing at least one portion of the aqueous feed and at least a portion of the organic feed to contact each other within the reaction column thereby converting at least a portion of the methyl chloride to methyl bromide.

22. A method of converting methyl chloride to methyl bromide comprising:
(a) providing a reaction column oriented substantially vertically along its axis and having an upper portion and a lower portion, wherein the reaction column contains a solid supported phase transfer catalyst;
(b) introducing an aqueous feed into the upper portion of the reaction column, wherein the aqueous feed comprises water and a bromide salt selected from the group consisting of alkali metal bromides, alkaline earth metal bromides and combinations thereof;
(c) introducing an organic feed into the lower portion of the reaction column, wherein the organic feed comprises methyl chloride and a water immiscible organic solvent; and
(d) allowing at least a portion of the aqueous feed and at least a portion of the organic feed to contact each other within the reaction column in the presence of the solid supported salt phase transfer catalyst, thereby converting at least a portion of the methyl chloride to methyl bromide.
